(19) United States
(12) Patent Application Publication
(10) Pub. No.: US 2026/0263409 A1
(43) Pub. Date: Sep. 10, 2026

(54) FLAVORED TOPICAL ANESTHETIC

(71) Applicant: Tamara Pesola, Mississauga (CA)

(72) Inventor: Tamara Pesola, Mississauga (CA)

(21) Appl. No.: 19/446,922

(22) Filed: Jan. 12, 2026

Related U.S. Application Data
(63) Continuation-in-part of application No. 19/028,610, filed on Jan. 17, 2025.

(51) Int. Cl. A61K 31/245; A61K 9/00; A61K 31/167; A61K 31/381; A61K 31/4453; A61K 31/47; A61K 47/10

(52) U.S. Cl. CPC A61K 31/245; A61K 9/0014; A61K 9/006; A61K 31/167; A61K 31/381; A61K 31/4453; A61K 31/47; A61K 47/10

(57) ABSTRACT

A topical anesthetic formulation topical analgesic formulation comprising an analgesic compound, at least one flavoring agent, and a cosmetically acceptable carrier, wherein the formulation is prepared for topical application to sensitive external skin and mucosal or mucosa-adjacent surfaces, including but not limited to the lips and perioral region.

### FIELD OF THE INVENTION

The present invention relates generally to topical anesthetics, more specifically but not by way of limitation, a topical anesthetic for use in providing anesthetic effects to sensitive external skin and mucosal or mucosa-adjacent surfaces, including but not limited to the lips and perioral region.

### BACKGROUND

Lip augmentation is a widely sought-after cosmetic procedure aimed at enhancing the fullness and shape of the lips. With its growing popularity, particularly in the beauty and aesthetic industries, ensuring patient comfort and minimizing procedural pain have become paramount. Numbing the lips prior to lip augmentation injections plays a critical role in achieving these goals, contributing not only to patient satisfaction but also to the overall success of the procedure. The application of a numbing agent prior to lip augmentation is a standard practice designed to mitigate the discomfort associated with the procedure. Lip injections typically involve the use of dermal fillers, such as hyaluronic acid-based products, which are administered via fine needles or cannulas. While these tools are designed to minimize trauma to the skin and underlying tissues, the sensitive nature of the lips can make the process uncomfortable. By applying a numbing agent, such as a topical anesthetic or a dental block, practitioners can significantly reduce the pain experienced during the injections.

Topical anesthetics are among the most commonly used methods for numbing the lips. These agents, which often include lidocaine or benzocaine, are applied directly to the lip area and left to penetrate the skin for a prescribed period. This approach is non-invasive and easy to administer, making it a convenient option for both practitioners and patients. However, the efficacy of topical anesthetics can vary based on factors such as the thickness of the lip tissue and the duration of application. In cases where more profound numbing is required, dental blocks may be employed. This technique involves the injection of a local anesthetic, such as lidocaine, into specific nerve sites to block pain signals. Although this method is more invasive than the application of topical agents, it offers a higher level of pain relief, making it suitable for patients with lower pain thresholds or for procedures involving extensive lip augmentation. The choice between topical anesthetics and dental blocks depends on the practitioner's assessment of the patient's needs and preferences.

Beyond physical comfort, numbing the lips also has psychological benefits for patients undergoing lip augmentation. The anticipation of pain can lead to heightened anxiety, which may negatively impact the patient's overall experience. By effectively numbing the treatment area, practitioners can alleviate these concerns, fostering a sense of trust and confidence in the procedure. This, in turn, can enhance the patient's perception of the results and encourage repeat visits for maintenance or additional cosmetic treatments. Moreover, minimizing pain during lip augmentation can contribute to better procedural outcomes. Pain-induced movements or flinching during injections can disrupt the precise placement of dermal fillers, potentially leading to uneven or suboptimal results. A numbed treatment area allows for greater control and accuracy during the procedure, ensuring that the desired aesthetic outcomes are achieved. Additionally, reduced pain may lead to a smoother recovery process, as patients are less likely to experience significant swelling or bruising related to procedural discomfort.

While numbing agents are highly effective, it is essential to consider potential risks and side effects. Topical anesthetics may cause mild irritation or allergic reactions in some patients, while dental blocks carry a small risk of nerve damage or prolonged numbness. Thorough patient assessment and a clear discussion of the benefits and risks associated with numbing techniques are crucial steps in the pre-procedure consultation process. This ensures that patients are well-informed and can make decisions that align with their comfort levels and aesthetic goals. Whether through the application of topical anesthetics or the use of dental blocks, this practice underscores the importance of patient-centered care in cosmetic medicine. Patients have expressed their dissatisfaction in regards to the poor taste of conventional numbing creams which provides a negative impact on the procedure.

Accordingly, there is a need for a dosage container and applicator for a formulation of topical anesthetic wherein the present invention includes a flavoring agent as a percentage of the formulation.

### SUMMARY OF THE INVENTION

It is the object of the present invention to provide proprietary formulation of a topical anesthetic for use in providing anesthetic effects to sensitive external skin and mucosal or mucosa-adjacent surfaces, including but not limited to the lips and perioral region.

The object of the present invention is to provide a topical anesthetic formulation comprising an analgesic compound, at least one flavoring agent and a cosmetically acceptable carrier.

The flavoring agent may be selected from the group consisting of: vanillin, ethyl vanillin, vanilla flavor systems, fruit flavors and non-menthol flavors. The analgesic compound may be selected from the group consisting of one or more of: lidocaine, benzocaine, tetracaine, prilocaine, dibucaine, dyclonine, articaine, and pharmaceutically acceptable salts thereof. The cosmetically acceptable carrier may comprise one or more emollients selected from the group consisting of: dimethicone, triglycerides, squalane, hydrogenated polyisobutene, alkyl benzoates, oils, waxes, butters, humectants, emulsifiers, and thickeners.

A further object of the present invention is to provide a formulation of topical anesthetic further comprising one or more additional sensory enhancement agents, the sensory enhancement agents selected from the group consisting of: flavor enhancers, sweeteners, aromatic additives, taste-smoothing agents, warming agents and cooling agents.

Yet a further object of the present invention is to provide a topical anesthetic formulation that is provided as a formulation selected from the group consisting of: creams, gels, lotions, balms, foams, sprays and wipes.

Still another object of the present invention is to provide a formulation of topical anesthetic wherein the formulation is packaged in a container selected from the group consisting of: tubes, jars, pump bottles, airless pumps, sachets, wipes, pads, vials and multi-dose containers.

An additional object of the present invention is to provide one or more methods of preparing a topical anesthetic formulation as described above.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### DETAILED DESCRIPTION

The present invention refers to embodiments of a topical anesthetic formulation and a method of preparing a topical anesthetic formulation according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

The topical anesthetic formulation comprises an analgesic compound, at least one flavoring agent, and a cosmetically acceptable carrier. The formulation is preferably prepared for topical application to sensitive external skin and mucosal or mucosa-adjacent surfaces, including but not limited to the lips and perioral region.

In an embodiment, the analgesic compound comprises 0.5%-30% of the formulation by weight, the flavoring agent comprises 0.5%-25% of the formulation by weight, and the carrier comprises 10%-95% of the formulation by weight.

The formulation is primarily intended for use in providing anesthetic effects to sensitive external skin and mucosal or mucosa-adjacent surfaces, notably the lips and perioral region. However, the formation may be suitable for topical application to any external skin or mucosal region of the body. Skin surfaces may include facial skin, tattooed skin, and body areas prepared for injections or cosmetic procedures. Mucosal surfaces may include lips, oral mucosa, perioral tissues, genital and anorectal regions.

As comprised, the flavoring agent acts to modify the user experience of the analgesic by masking any bitterness or other harsh taste associated with topical anesthetics, by improving taste, aroma, mouthfeel, and overall organoleptic properties of the formulation, and/or improving tolerability and user acceptance of the formulation.

The formulation is designed for use before, during and/or after cosmetic, dermatologic, aesthetic, or personal-care procedures, and/or for further pain relief in other non-procedural contexts. Exemplary procedures may include cosmetic and aesthetic injections (e.g., dermal fillers, neuromodulators), permanent makeup and lip blushing, body and facial tattooing, microneedling and laser treatments, piercing (including lip piercing), and dental-adjacent applications where topical anesthetics are used on mucosal or perioral tissue.

The formation may also be used for over-the-counter (OTC) uses such as temporary relief of pain, itching, burning, or discomfort in anorectal or other topical indications.

The formulation may further comprise one or more additional sensory enhancement agents, the sensory enhancement agents may be selected from the group consisting of: flavor enhancers (as esters, aldehydes, ketones, and other recognized flavoring agents), sweeteners (e.g. sucralose, xylitol, sorbitol, stevia, glycerin, ethyl maltol, honey derivatives, and other pharmaceutically acceptable sweeteners or taste enhancers), aromatic additives, such as essential oils and natural aromatics (e.g., peppermint, spearmint, wintergreen, eucalyptus, lavender, chamomile, citrus oils, vanilla, cocoa, fruit oils), taste-smoothing agents, warming or cooling agents (menthol, eucalyptol, vanillyl butyl ether, capsaicin analogs, pepper extracts). The additional sensory enhancement agents may comprise 0.01%-10% of the formulation by weight.

The formulation is preferably compounded as a gel or cream, however, the formulation may be compounded as a lotion, balm, foam, spray or provided on a wipe. However, the formulation is generally not suitable for use in a dissolving strip or equivalent, an aerosol delivery system, an injectable delivery system or as a transdermal reservoir patch.

Once compounded, the formulation may be packaged for storage and dispensing in a suitable container such as a tube, jar, pump bottle, airless pump, sachet, wipe, pad, or vial and may be provided in single-dose or multi-dose containers.

The analgesic compound may be formed from a single analgesic or multiple analgesic and may be selected from one or more of: lidocaine, benzocaine, tetracaine, prilocaine, dibucaine, dyclonine, articaine, and pharmaceutically acceptable salts thereof.

The flavoring agent may be formed from a single agent or multiple agents and may be selected from one or more of: vanillin, ethyl vanillin, vanilla flavor systems, fruit flavors and non-menthol flavors. The final formulation may have a specific flavor or sensory profile, such as vanilla-mint, strawberry-cream, citrus-cool, warm vanilla, or mint-chocolate.

The cosmetically acceptable carrier may be formed from a single carrier or multiple carriers, preferably one or more emollients, and selected from one or more of: dimethicone, triglycerides, squalane, hydrogenated polyisobutene, alkyl benzoates, oils, waxes, butters, humectants, emulsifiers, and thickeners.

One non-limiting exemplary formulation may be provided as a low-strength analgesic. The low-strength formulation may comprise approximately 4% by weight of lidocaine, combined with the following elements: caprylic/capric triglyceride, hydrogenated polyisobutene, C12-15 alkyl benzoate, beeswax and/or fatty alcohols (e.g., cetearyl alcohol), plant butters or oils (e.g., jojoba oil, shea butter), silicones (e.g., dimethicone), texture modifiers (e.g., silica), antioxidants (e.g., tocopheryl acetate), and the flavoring agent. The formulation may also comprise one or more sensory enhancement agents including flavor enhancers, sweetening/taste-masking agents, and/or aromatic agents (e.g., vanilla-type components).

A similar formulation may be used to provide a medium-strength analgesic, comprising approximately 7% lidocaine by weight, combined with similar elements.

A similar, still stronger formulation may be provided with a benzocaine/lidocaine/tetracaine (BLT) analgesic compound at approximately 30% by weight, or a single analgesic, such as lidocaine at 20%-23% by weight. Again, the analgesic compound may then be combined with similar elements to produce the final formulation.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A topical analgesic formulation comprising:
an analgesic compound,
at least one flavoring agent, and
a cosmetically acceptable carrier,
wherein the formulation is prepared for topical application to sensitive external skin and mucosal or mucosa-adjacent surfaces, including but not limited to the lips and perioral region.

2. The topical analgesic formulation as recited in claim 1, further comprising one or more additional sensory enhancement agents comprising 0.01%-10% of the formulation by weight, the sensory enhancement agents selected from the group consisting of: flavor enhancers, sweeteners, aromatic additives, taste-smoothing agents, warming agents and cooling agents.

3. The topical analgesic formulation as recited in claim 1, wherein the flavoring agent is selected from the group consisting of one or more of: vanillin, ethyl vanillin, vanilla flavor systems, fruit flavors and non-menthol flavors.

4. The topical analgesic formulation as recited in claim 1, wherein the analgesic compound comprises 0.5%-30% of the formulation by weight, the analgesic compound selected from the group consisting of one or more of: lidocaine, benzocaine, tetracaine, prilocaine, dibucaine, dyclonine, articaine, and pharmaceutically acceptable salts thereof.

5. The topical analgesic formulation as recited in claim 1, wherein the formulation is selected from the group consisting of: creams, gels, lotions, balms, foams, sprays and wipes.

6. The topical analgesic formulation as recited in claim 1, wherein the formulation is packaged in a container selected from the group consisting of: tubes, jars, pump bottles, airless pumps, sachets, wipes, pads, vials and multi-dose containers.

7. The topical analgesic formulation as recited in claim 1, wherein the cosmetically acceptable carrier comprises 10%-95% of the formulation by weight and the cosmetically acceptable carrier further comprises one or more emollients selected from the group consisting of: dimethicone, triglycerides, squalane, hydrogenated polyisobutene, alkyl benzoates, oils, waxes, butters, humectants, emulsifiers, and thickeners.

8. A method of preparing a topical analgesic formulation, comprising:
combining an analgesic compound, at least one flavoring agent, and a cosmetically acceptable carrier into the topical analgesic formulation, the topical analgesic formulation being selected from the group consisting of: creams, gels, lotions, balms, foams, sprays and wipes
wherein the formulation is prepared for topical application to sensitive external skin and mucosal or mucosa-adjacent surfaces, including but not limited to the lips and perioral region.

9. The method as recited in claim 8, further comprising combining one or more additional sensory enhancement agents into the topical analgesic formulation, the sensory enhancement agents selected from the group consisting of: flavor enhancers, sweeteners, aromatic additives, taste-smoothing agents, warming agents and cooling agents.

10. The method as recited in claim 8, wherein the flavoring agent is selected from the group consisting of one or more of: vanillin, ethyl vanillin, vanilla flavor systems, fruit flavors and non-menthol flavors.

11. The method as recited in claim 8, wherein the analgesic compound is selected from the group consisting of one or more of: lidocaine, benzocaine, tetracaine, prilocaine, dibucaine, dyclonine, articaine, and pharmaceutically acceptable salts thereof.

12. The method as recited in claim 8, wherein the cosmetically acceptable carrier comprises one or more emollients selected from the group consisting of: dimethicone, triglycerides, squalane, hydrogenated polyisobutene, alkyl benzoates, oils, waxes, butters, humectants, emulsifiers, and thickeners.

13. The method as recited in claim 8, further comprising packaging the formulation in a container selected from the group consisting of: tubes, jars, pump bottles, airless pumps, sachets, wipes, pads, vials and multi-dose containers.
